(19) United States
(12) Patent Application Publication
(10) Pub. No.: US 2015/0025380 A1
(43) Pub. Date: Jan. 22, 2015

(54) ULTRASOUND DIAGNOSIS APPARATUS, IMAGE PROCESSING APPARATUS AND IMAGE PROCESSING METHOD

(71) Applicants: Kabushiki Kaisha Toshiba, Minato-ku (JP); Toshiba Medical Systems Corporation, Otawara-shi (JP)

(72) Inventors: Mio AZEGAMI, Nasushiobara-shi (JP); Masao Takimoto, Otawara-shi (JP); Naohisa Kamiyama, Utsunomiya (JP)

(73) Assignees: Kabushiki Kaisha Toshiba, Minato-ku (JP); Toshiba Medical Systems Corporation, Otawara-shi (JP)

(21) Appl. No.: 14/509,110

(22) Filed: Oct. 8, 2014

Related U.S. Application Data
(63) Continuation of application No. PCT/JP2013/061227, filed on Apr. 15, 2013.

(30) Foreign Application Priority Data
Apr. 13, 2012 (JP) 2012-092354

(51) Int. Cl. A61B 8/08; A61B 8/14; A61B 8/00; A61B 8/06

(52) U.S. Cl. CPC A61B 8/085; A61B 8/06; A61B 8/14; A61B 8/488; A61B 8/463
USPC 600/438

(57) ABSTRACT

An ultrasound diagnosis apparatus according to an embodiment includes a transmitting and receiving unit, a detecting unit, and a chart display unit. The transmitting and receiving unit transmits an ultrasound pulse to a subject and receives an echo signal from the subject. The detecting unit detects thrombi that are present in the blood of the subject from the echo signal and generates numerical value information that quantifies a thrombus detection result. The chart display unit displays a chart indicating the thrombus detection result on a display unit, on the basis of the numerical value information.

### CROSS-REFERENCE TO RELATED APPLICATIONS

This application is a continuation of PCT international application Ser. No. PCT/JP2013/061227 filed on Apr. 15, 2013 which designates the United States, incorporated herein by reference, and which claims the benefit of priority from Japanese Patent Application No. 2012-092354, filed on Apr. 13, 2012, the entire contents of which are incorporated herein by reference.

### FIELD

Embodiment described herein relates generally to an ultrasound diagnosis apparatus, an image processing apparatus and an image processing method.

### BACKGROUND

In recent years, treatment methods are known by which an auxiliary artificial heart is installed in a subject's body and by which a stent is held in place. When those treatment methods are implemented, it is considered that thrombi are more easily formed due to immune reactions or the like. Because thrombi can cause thrombotic embolism and the like, controlling thrombi is important.

In this regard, examples of methods that are conventionally known include: a method by which thrombi are monitored by inserting a transesophageal probe into a subject's body; and a method by which thrombi are monitored by adhering an ultrasound sensor to an inflow port and/or an outflow port of an auxiliary artificial heart.

### BRIEF DESCRIPTION OF THE DRAWINGS

FIG. 1 is a diagram of an ultrasound diagnosis apparatus according to an embodiment;

FIG. 2 is a drawing for explaining an approach used by an ultrasound probe according to the present embodiment;

FIG. 3 is a drawing of Chart 1 according to the present embodiment;

FIG. 4A is a drawing of a modification example of Chart 1 according to the present embodiment;

FIG. 4B is a drawing of another modification example of Chart 1 according to the present embodiment;

FIG. 5 is a drawing of Chart 2 according to the present embodiment;

FIG. 6 is a drawing of Chart 3 according to the present embodiment;

FIG. 7 is a drawing of Chart 4 according to the present embodiment;

FIG. 8 is a drawing of Chart 5 according to the present embodiment;

FIG. 9 is another drawing of Chart 5 according to the present embodiment;

FIG. 10 is yet another drawing of Chart 5 according to the present embodiment; and

FIG. 11 is a diagram of an image processing apparatus according to another embodiment.

### DETAILED DESCRIPTION

An ultrasound diagnosis apparatus according to an embodiment includes a transmitting and receiving unit, a detecting unit, and a chart display unit. The transmitting and receiving unit transmits an ultrasound pulse to a subject and receives an echo signal from the subject. The detecting unit detects thrombi that are present in the blood of the subject from the echo signal and generates numerical value information that quantifies a thrombus detection result. The chart display unit displays a chart indicating the thrombus detection result on a display unit, on the basis of the numerical value information.

Ultrasound diagnosis apparatuses are medical image diagnosis apparatuses configured to display images of tissues inside the body of an examined subject. Compared to other medical image diagnosis apparatuses such as X-ray diagnosis apparatuses and X-ray Computed Tomography (CT) apparatuses, ultrasound diagnosis apparatuses are less expensive and involve no dose of radiation and are therefore utilized as useful apparatuses for making observations in a non-invasive and real-time manner. Ultrasound diagnosis apparatuses have a wide range of applications and can be used for circulatory organs such as the heart, abdominal parts including the liver and the kidneys, and peripheral blood vessels, as well as diagnoses in obstetrics and gynecology and for breast cancer. In particular, because of the characteristic of ultrasound diagnosis apparatuses that enables the user to observe a subject in a real-time manner with a high frame rate, ultrasound diagnosis apparatuses are excellent for the purpose of making dynamic diagnoses of an organ and are therefore excellent for the purpose of making diagnoses such as abnormalities in the wall motions of the heart. Further, a Doppler method, which is implemented as an exemplary function of ultrasound diagnoses, makes it possible to extract bloodstream and to represent the extracted bloodstream in an image and is therefore highly effective in dynamic diagnoses of bloodstream in any diagnosed regions.

Incidentally, when a severe heart failure has occurred, because the heart is no longer able to perform the pumping function sufficiently, it is considered that a heart transplant is beneficial. However, only approximately 10 percent of the subjects in the stand-by list world-wide actually receive heart transplants. Also, not a small number of subjects are evaluated as not being suitable for a transplant. To cope with these situations, treatments using auxiliary artificial hearts are performed, by which it is possible to recover the cardiac functions while reducing loads on the heart. When a left ventricular assist device is used, blood is drained from the left ventricular apex and is caused to flow into the aorta via a pump. As a result, it is possible to maintain and enhance the circulation in the subject's body without putting a load on the heart of the subject. After an auxiliary artificial heart is installed, thrombi can be very much more easily formed due to bacteremia caused by a bacterial infection after the installation, inflammation caused by an immune reaction, blood stagnation inside the pump, the generation of heat by the pump, and mechanical contact. Thrombi caused by these factors can also be a cause of clogging in capillaries. Thus, it is important to control thrombi after the installation of an auxiliary artificial heart.

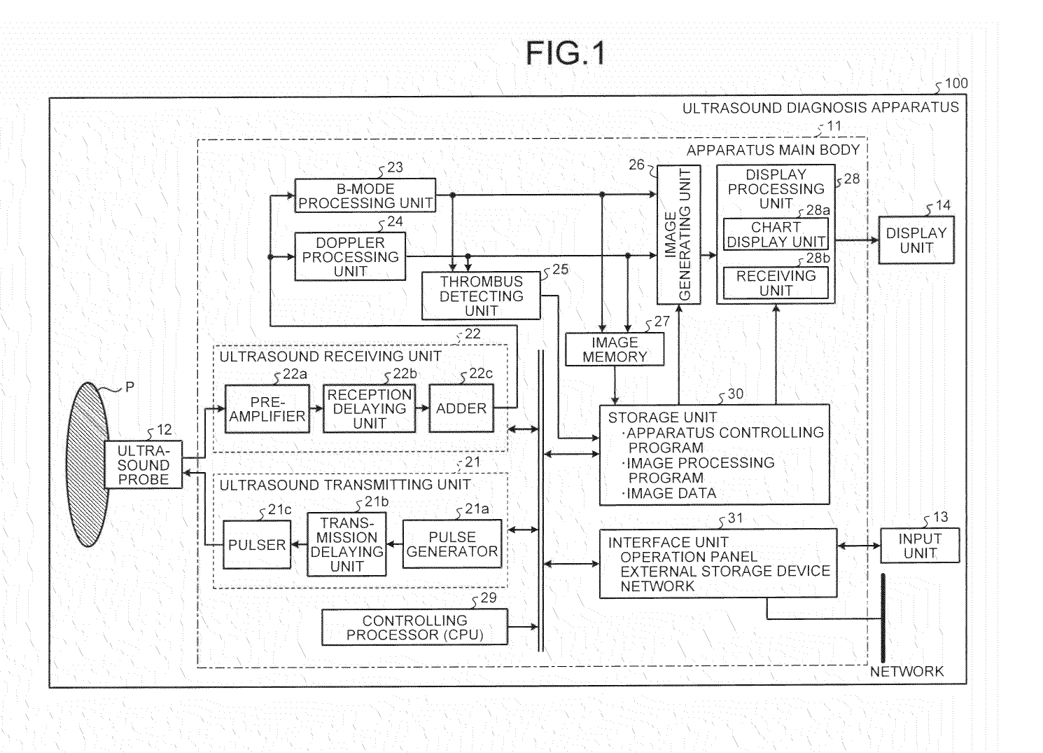

FIG. 1 is a diagram of an ultrasound diagnosis apparatus 100 according to an embodiment. As shown in FIG. 1, the ultrasound diagnosis apparatus 100 according to the present embodiment includes an apparatus main body 11, an ultrasound probe 12, an input unit 13, and a display unit 14. The ultrasound diagnosis apparatus 100 does not include an examined subject P and a network.

The ultrasound probe 12 includes a plurality of piezoelectric transducer elements. The plurality of piezoelectric transducer elements generate an ultrasound pulse based on a drive signal supplied from an ultrasound transmitting unit 21 (explained later). Further, the plurality of piezoelectric transducer elements receive a reflected wave from the subject P and convert the received reflected wave into an electric signal. Further, the ultrasound probe 12 includes matching layers included in the piezoelectric transducer elements, as well as a backing member that prevents ultrasound waves from propagating rearward from the piezoelectric transducer elements.

When an ultrasound pulse is transmitted from the ultrasound probe 12 to the subject P, the transmitted ultrasound pulse is repeatedly reflected on a surface of discontinuity of acoustic impedances at a tissue in the body of the subject P and is received as an echo signal by the plurality of piezoelectric transducer elements included in the ultrasound probe 12. The amplitude of the received echo signal is dependent on the difference between the acoustic impedances on the surface of discontinuity on which the ultrasound pulse is reflected. When the transmitted ultrasound pulse is reflected on the surface of a flowing bloodstream or a cardiac wall, the echo signal is, due to the Doppler effect, subject to a frequency shift, depending on a velocity component of the moving members with respect to the ultrasound wave transmission direction.

The input unit 13 includes a mouse, a keyboard, a button, a panel switch, a touch command screen, a foot switch, a trackball, and/or the like and is connected to the apparatus main body 11. The input unit 13 receives various types of instructions and setting requests from an operator of the ultrasound diagnosis apparatus 100 and transfers the received various types of instructions and setting requests to the apparatus main body 11. For example, the input unit 13 receives a setting request to set a Region Of Interest (ROI) from the operator and transfers the received ROI to the apparatus main body 11.

The display unit 14 displays a Graphical User Interface (GUI) used by the operator of the ultrasound diagnosis apparatus 100 to input the various types of instructions and setting requests through the input unit 13. Further, the display unit 14 displays an ultrasound image generated by an image generating unit 26 (explained later). The display unit 14 also displays a chart generated by a chart display unit 28a (explained later). Examples of the ultrasound image include a B-mode image, an M-mode image, and a Doppler image (e.g., a color Doppler image or a pulse Doppler image), which represents morphological information and/or bloodstream information of the subject.

The apparatus main body 11 includes the ultrasound transmitting unit 21, an ultrasound receiving unit 22, a B-mode processing unit 23, a Doppler processing unit 24, a thrombus detecting unit 25, the image generating unit 26, an image memory 27, a display processing unit 28, a controlling processor (a Central Processing Unit (CPU)) 29, a storage unit 30, and an interface unit 31. The ultrasound transmitting unit 21, the ultrasound receiving unit 22, and the like that are included in the apparatus main body 11 may be configured by using hardware such as an integrated circuit or may be configured by using a computer program.

The ultrasound transmitting unit 21 is configured to transmit the ultrasound pulse to the subject P. More specifically, the ultrasound transmitting unit 21 includes a pulse generator 21a, a transmission delaying unit 21b, a pulser 21c, and the like and supplies the drive signal to the ultrasound probe 12. The pulse generator 21a repeatedly generates a rate pulse for forming the ultrasound pulse at a predetermined Pulse Repetition Frequency (PRF). The PRF may also be referred to as a rate frequency (frHz). Further, the transmission delaying unit 21b applies a transmission delay period that is required to converge the ultrasound pulse generated by the ultrasound probe 12 into the form of a beam and to determine transmission directionality and that corresponds to each of the piezoelectric transducer elements, to each of the rate pulses generated by the pulse generator 21a. Further, the pulser 21c applies the drive signal (a drive pulse) to the ultrasound probe 12 with timing based on the rate pulses. In other words, the transmission delaying unit 21b arbitrarily adjusts the directions of the transmissions from the piezoelectric transducer element surfaces, by varying the transmission delay periods applied to the rate pulses.

The ultrasound receiving unit 22 is configured to receive the echo signal from the subject P. More specifically, the ultrasound receiving unit 22 includes a pre-amplifier 22a, a reception delaying unit 22b, an adder 22c, and the like and is configured to perform various types of processes on the echo signal received by the ultrasound probe 12. The pre-amplifier 22a is configured to amplify the echo signal for each of the channels and to perform a gain correcting process. An analog/digital (A/D) converter (not shown) applies an A/D conversion to the gain-corrected echo signal. The reception delaying unit 22b applies a reception delay period required to determine reception directionality to the echo signal. The adder 22c performs an adding process on the echo signals to which the reception delay periods were applied by the reception delaying unit 22b. As a result of the adding process performed by the adder 22c, reflected components from the direction corresponding to the reception directionality of the echo signals are emphasized. A comprehensive reception beam for the ultrasound transmission/reception is thus formed according to the reception directionality and the transmission directionality. The form of the echo signals output from the ultrasound receiving unit 22 may be selected from various types of forms and may be in the form of signals called Radio Frequency (RF) signals that contain phase information or may represent amplitude information obtained after an envelope detection process is performed.

The B-mode processing unit 23 is configured to receive the echo signals from the ultrasound receiving unit 22 and to generate B-mode data by performing a logarithmic amplification, an envelope detection process, and the like on the received echo signals. The B-mode data is data from scanning lines in which the strength of each echo signal is expressed by a degree of brightness. The B-mode processing unit 23 sends the generated B-mode data to the thrombus detecting unit 25 and to the image generating unit 26.

The Doppler processing unit 24 is configured to receive the echo signals from the ultrasound receiving unit 22 and to generate Doppler data by performing, for example, a frequency analysis so as to obtain a velocity of the bloodstream. The Doppler data is data obtained by extracting bloodstream, tissues, and contrast echo components that are under the influence of the Doppler effect and extracting moving member information such as an average velocity, a dispersion, a power, and the like for a plurality of points. The Doppler processing unit 24 sends the generated Doppler data to the thrombus detecting unit 25 and to the image generating unit 26.

The thrombus detecting unit 25 is configured to detect thrombi, if any, that are present in the blood of the subject P from the echo signals and to generate numerical value information that quantifies the thrombus detection result. More specifically, the thrombus detecting unit 25 receives either the B-mode data or the Doppler data from either the B-mode processing unit 23 or the Doppler processing unit 24, performs an analyzing process to detect the thrombi on either the received B-mode data or the received Doppler data, and detects the thrombi that are present in the blood of the subject P. Further, the thrombus detecting unit 25 generates the numerical value information that quantifies the thrombus detection result and sends the generated numerical value information to the storage unit 30. The details of the process performed by the thrombus detecting unit 25 will be explained later.

The image generating unit 26 is configured to generate an ultrasound image from the B-mode data generated by the B-mode processing unit 23 or the Doppler data generated by the Doppler processing unit 24. More specifically, the image generating unit 26 converts (by performing a scan convert process) data from a plurality of scanning lines contained in the B-mode data into a scanning line data that is in a video format used by, for example, television and generates the ultrasound image serving as a displayed image.

For example, the image generating unit 26 generates a B-mode image from the B-mode data. As another example, the image generating unit 26 generates a Doppler image from the Doppler data, the Doppler image being, for example, an average velocity image, a dispersion image, a power image, or an image combining any of these. The data before being input to the image generating unit 26 may be referred to as “raw data”.

The image memory 27 stores therein the B-mode data generated by the B-mode processing unit 23, the Doppler data generated by the Doppler processing unit 24, the ultrasound image generated by the image generating unit 26, and the like. After a diagnosis process, for example, the operator is able to invoke one or more images that were recorded during a medical examination and to play back each image in the manner of a still image or play back multiple images in the manner of a moving picture.

The display processing unit 28 is configured to perform various types of processes such as a correcting process to correct a dynamic range, brightness levels, a contrast, or a γ curve or an RGB conversion process, on the ultrasound image generated by the image generating unit 26 and to cause the ultrasound image resulting from the performed process to be displayed on the display unit 14. For example, the display processing unit 28 causes the B-mode image generated by the image generating unit 26 to be displayed on the display unit 14. In another example, the display processing unit 28 causes the Doppler image generated by the image generating unit 26 to be displayed on the display unit 14 in color. Further, the display processing unit 28 according to the present embodiment includes the chart display unit 28a and a receiving unit 28b.

On the basis of the numerical value information generated by the thrombus detecting unit 25, the chart display unit 28a displays a chart indicating the thrombus detection result on the display unit 14. More specifically, when the chart display unit 28a has obtained the numerical value information generated by the thrombus detecting unit 25 by referring to the storage unit 30, the chart display unit 28a generates, as appropriate, the chart indicating the thrombus detection result on the basis of the obtained numerical value information, and displays the generated chart on the display unit 14. The details of the process performed by the chart display unit 28a will be explained later.

When the B-mode image generated by the image generating unit 26 is caused to be displayed on the display unit 14 by the display processing unit 28, the receiving unit 28b receives a designation of an analysis region in the B-mode image. In this situation, for example, the thrombus detecting unit 25 detects thrombi, if any, within the analysis region received by the receiving unit 28b. Further, for example, the Doppler processing unit 24 outputs a Doppler waveform indicating a flow rate of the bloodstream in the analysis region received by the receiving unit 28b.

The controlling processor 29 has a function of an information processing apparatus (a computer) and is configured to control overall processes performed by the ultrasound diagnosis apparatus 100. More specifically, on the basis of the various types of instructions and setting requests input by the operator via the input unit 13, as well as various types of computer programs and various types of setting information read from the storage unit 30, the controlling processor 29 controls processes performed by the ultrasound transmitting unit 21, the ultrasound receiving unit 22, the B-mode processing unit 23, the Doppler processing unit 24, the thrombus detecting unit 25, the image generating unit 26, and the display processing unit 28 and also exercises control so as to cause the ultrasound image and the like stored in the image memory 27 to be displayed on the display unit 14.

For example, the controlling processor 29 detects the thrombi from the echo signals, reads a computer program (hereinafter, an “image processing program”) dedicated for displaying a chart from the storage unit 30, and performs computations and exercises control related to various types of processes. Further, for example, the controlling processor 29 reads a transmission delay pattern and a reception delay pattern stored in the storage unit 30 and switches between a transmission delay and a reception delay, in accordance with the transmission direction and the reception direction.

The storage unit 30 stores therein: an apparatus controlling computer program for performing the ultrasound transmission and reception, the image processing process, and the display processing process; the computer program (the “image processing program”) dedicated for detecting the thrombi from the echo signals and for displaying the chart; diagnosis information (e.g., subjects' IDs, medical doctors' observations); and various types of data such as diagnosis protocols and various types of setting information. Further, the storage unit 30 may be used, as necessary, for storing therein any of the images stored in the image memory 27. Further, it is possible to transfer the data stored in the storage unit 30 to an external peripheral apparatus via the interface unit 31.

The interface unit 31 is an interface related to the input unit 13 and an external storage device (not shown). It is possible to transfer data such as the ultrasound image and the analysis result obtained by the ultrasound diagnosis apparatus 100 to another apparatus via the interface unit 31 through a network.

Next, details of a process related to the thrombus detecting process according to the present embodiment will be explained. In the present embodiment, an example will be explained in which one of the purposes is to understand a thrombus formation state of a subject who has an auxiliary artificial heart has installed. However, possible embodiments are not limited to this example.

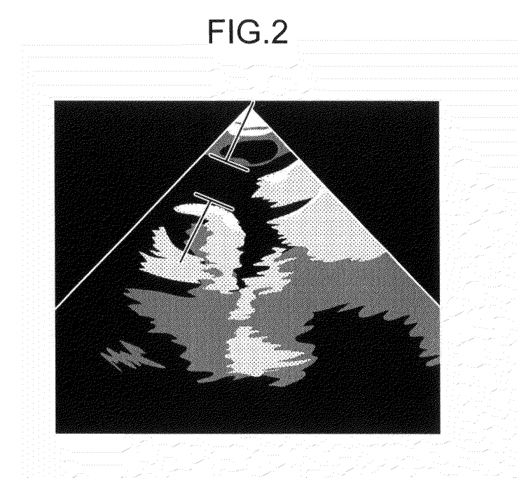

FIG. 2 is a drawing for explaining an approach used by the ultrasound probe 12 according to the present embodiment. In the present embodiment, the ultrasound diagnosis apparatus 100 is configured to detect thrombi, if any, at the aortic arch, while using an approach by which the ultrasound probe 12 is placed on the suprasternal notch, without being inserted into the body of the subject. FIG. 2 illustrates an ultrasound image obtained by using the approach by which the ultrasound probe 12 is placed on the suprasternal notch. The aortic arch is a site at which the circulation flowing from the subject's heart to the aorta meets a bypass circulation flowing from an apex to the aorta via the auxiliary artificial heart. The aortic arch corresponds to a source of the body circulation. Thrombi that are detected at the aortic arch will flow to the brain and peripherals such as the arms and the legs. For this reason, generating the numerical value information that quantifies the thrombus detection result detected at the aortic arch and displaying the chart indicating the thrombus detection result, as described in the present embodiment, are helpful and useful for considering different treatment plans. In the present embodiment, the approach by which the ultrasound probe 12 is placed on the suprasternal notch is explained as an example; however, possible embodiments are not limited to this example.

Next, details of a process performed by the thrombus detecting unit 25 will be explained. As described above, the thrombus detecting unit 25 is configured to perform the analyzing process on either the B-mode data or the Doppler data and to detect the thrombi that are present in the blood of the subject P. The analyzing process may be realized by using a publicly-known technique, for example. In the present embodiment, an example will be explained in which the analyzing process is performed on either the B-mode data or the Doppler data. However, possible embodiments are not limited to this example. For example, the thrombus detecting unit 25 may perform the analyzing process on a B-mode image, an M-mode image, or a Doppler image (e.g., a color Doppler image or a pulse Doppler image) generated by the image generating unit 26.

Generally speaking, it is difficult to detect thrombi from B-mode data, because the acoustic impedances of red blood cells and white blood cells are similar to that of blood plasma. However, the acoustic impedances of thrombi are relatively high. For this reason, it is possible to detect thrombi, if any, from the B-mode data. Further, in the Doppler data, the power of a Doppler component of a thrombus momentarily exhibits a significantly high signal. For this reason, it is also possible to detect thrombi, if any, from the Doppler data. In the following sections, a first method by which thrombi are detected from B-mode data (for a B-mode image), a second method by which thrombi are detected from B-mode data (for an M-mode image), and a third method by which thrombi are detected from Doppler data, will be sequentially explained. Methods that can be used for detecting the thrombi are not limited to the first to the third methods described below. It is acceptable to employ any other publicly-known techniques.

First, an example of the first method by which thrombi are detected from B-mode data (for a B-mode image) will be explained. When the ultrasound probe 12 is placed on the suprasternal notch of the subject, the echo signals received by the ultrasound receiving unit 22 are sent to the B-mode processing unit 23, so that a B-mode image generated by the image generating unit 26 is displayed on the display unit 14 by the display processing unit 28. After that, the receiving unit 28b first receives a setting of an analysis region within the B-mode image displayed on the display unit 14. For example, as shown in FIG. 2, the receiving unit 28b receives a setting of a range gate within the B-mode image displayed on the display unit 14. The thrombus detecting unit 25 detects the thrombi within this analysis region.

Let us assume that the ultrasound probe 12 continues to be placed on the suprasternal notch of the subject so that B-mode data is acquired in a real-time manner. In this situation, the thrombus detecting unit 25 detects the thrombi by performing an analyzing process that uses a Fiber Structure Extraction Technique (FSET), on the B-mode data received from the B-mode processing unit 23.

More specifically, a pattern called a speckle pattern occurs in the B-mode data where speckles are distributed spatially at random. Further, an amplitude probability density distribution of speckle pattern signals can be approximated by a Rayleigh distribution. Thus, the thrombus detecting unit 25 judges whether each of the pixels in the B-mode data is a component that can be approximated by a Rayleigh distribution and detects non-Rayleigh components, which cannot be approximated by a Rayleigh distribution, as thrombi. For example, the thrombus detecting unit 25 first stores therein the brightness level of a pixel serving as a processing target. Subsequently, the thrombus detecting unit 25 calculates an average value of the brightness levels in the surroundings of the pixel serving as the processing target and further calculates the difference by subtracting the average value from the brightness level of the pixel serving as the processing target. The thrombus detecting unit 25 repeatedly performs this calculation for each of all the pixels in the B-mode data and subsequently performs reverse logarithmic transformation. After performing the reverse logarithmic transformation, the thrombus detecting unit 25 judges, for each of the pixels, whether the component can be approximated by a Rayleigh distribution or not by using a threshold value. The threshold value may be dynamically set for each of the processed pixels in the B-mode data, by using an expression defining a Rayleigh distribution as a criterion.

Subsequently, the thrombus detecting unit 25 generates the numerical value information by quantifying the thrombus detection result detected in the manner described above. For example, when the first method is used, the thrombus detecting unit 25 defines a block made up of pixels that are extracted as non-Rayleigh components and that are in continuity in the B-mode data, as one thrombus. The thrombus detecting unit 25 then calculates the quantity of thrombi (each of which is represented by a block of pixels), an area of each of the thrombi (the quantity of pixels contained in each of the blocks), and a total area of all the thrombi (the total value of the areas of the thrombi), for each unit time period. After that, the thrombus detecting unit 25 generates the numerical value information for each unit time period that presents the information about the quantity of thrombi as “the quantity of thrombi”, presents the information about the area of each of the thrombi as “the size of each of the thrombi”, and presents the information about the total area of all the thrombi as “the amount of thrombi”. The numerical value information obtained in this manner is stored into the storage unit 30 and is used in a process performed by the chart display unit 28a.

Next, an example of the second method by which thrombi are detected from B-mode data (for an M-mode image) will be explained. The analysis region setting process may be performed in the same manner as in the first method. Thus, the explanation thereof will be omitted.

An M-mode image represents temporal changes in data from a certain scanning line that is among the data from a plurality of scanning lines contained in a B-mode image. In other words, the B-mode data for an M-mode image is time-series data from a certain scanning line. As described above, in the data from the scanning lines, the strength of each echo signal is expressed by a degree of brightness. Also, as described above, the acoustic impedances of thrombi are relatively high. Thus, when a thrombus passes over a scanning line, a change occurs in the brightness level. For this reason, the thrombus detecting unit 25 analyzes the data from the scanning lines and detects pixels each having a brightness level that exceeds a predetermined threshold value as thrombi. Further, on the basis of continuity on each scanning line (the pixels each having a brightness level that exceeds the threshold value are in continuity on one scanning line) and on the basis of sequentiality along the time-series direction with respect to a plurality of scanning lines arranged in a time series (the pixels each having a brightness level that exceeds the threshold value are sequential along the time-series direction), the thrombus detecting unit 25 identifies each block of pixels and defines each block of pixels as one thrombus.

Subsequently, the thrombus detecting unit 25 generates the numerical value information by quantifying the thrombus detection result detected in the manner described above. For example, when the second method is used, the thrombus detecting unit 25 defines a block made up of pixels that are in continuity on one scanning line and are sequential along the time-series direction with respect to a plurality of scanning lines arranged in the time series, as one thrombus. The thrombus detecting unit 25 further calculates the quantity of thrombi (each of which is represented by a block of pixels), an area of each of the thrombi (the quantity of pixels contained in each of the blocks of pixels), and a total area of all the thrombi (the total value of the areas of the thrombi), for each unit time period. After that, the thrombus detecting unit 25 generates the numerical value information for each unit time period that presents the information about the quantity of thrombi as “the quantity of thrombi”, presents the information about the area of each of the thrombi as “the size of each of the thrombi”, and presents the information about the total area of all the thrombi as “the amount of thrombi”. The numerical value information obtained in this manner is stored into the storage unit 30 and is used in a process performed by the chart display unit 28a.

The method for detecting the thrombi from the B-mode data for the M-mode image is not limited to the second method. It is acceptable to use, alone or in combination, the method by which non-Rayleigh components are extracted as thrombi like in the first method.

Next, an example of the third method by which thrombi are detected from Doppler data will be explained. The analysis region setting process may be performed in the same manner as in the first method. Thus, the explanation thereof will be omitted.

In Doppler data, the power of a Doppler component is expressed by a degree of brightness. Further, as described above, the power of a Doppler component of a thrombus momentarily exhibits a significantly high signal. Thus, the thrombus detecting unit 25 analyzes the Doppler data and, if the power of a Doppler component exceeds a predetermined threshold value, the thrombus detecting unit 25 detects the Doppler component as a thrombus.

Subsequently, the thrombus detecting unit 25 generates the numerical value information by quantifying the thrombus detection result detected in this manner. For example, when the third method is used, the thrombus detecting unit 25 counts the number of times the power of a Doppler component exceeds the threshold value as the quantity of thrombi for each unit time period. After that, the thrombus detecting unit 25 generates the numerical value information for each unit time period that presents the information about the quantity of thrombi as “the quantity of thrombi”. The numerical value information obtained in this manner is stored into the storage unit 30 and is used in a process performed by the chart display unit 28a.

The method for detecting the thrombi from the Doppler data is not limited to the third method. For example, it is acceptable to detect the thrombi from dispersion information contained in the Doppler data. Further, the methods for detecting thrombi from the Doppler data may be applied to both a color Doppler image and a pulse Doppler image.

The processes described above that are performed by the thrombus detecting unit 25 may be performed in a real-time manner together with a real-time acquisition of the B-mode data or the Doppler data or may be performed afterwards.

Next, details of a process performed by the chart display unit 28a will be explained. On the basis of the numerical value information generated by the thrombus detecting unit 25, the chart display unit 28a displays a chart indicating the thrombus detection result on the display unit 14. By referring to the storage unit 30, the chart display unit 28a may generate, as appropriate, a chart that is suitable for the mode of operation, while selecting any parts of the numerical value information generated by the thrombus detecting unit 25 or processing the numerical value information (e.g., performing a separate calculation process thereon), as necessary. In other words, the chart display unit 28a does not necessarily need to generate all the charts described below. Further, the charts described below are merely examples. The chart display unit 28a may generate and display charts other than those described below. It may also be arbitrarily determined whether each of the charts has scales on the vertical and the horizontal axes.

For example, the chart display unit 28a displays the following charts:

Chart 1: a chart that indicates temporal changes in a distribution of sizes of thrombi and the quantity of thrombi for each size;

Chart 2: a chart that indicates temporal changes in the quantity of thrombi (or the amount of thrombi);

Chart 3: a chart that indicates temporal changes in an accumulated value of quantities of thrombi (or amounts of thrombi);

Chart 4: a chart that indicates a correlation between a Doppler waveform and various types of thrombus information; and

Chart 5: a chart that combines various types of charts together.

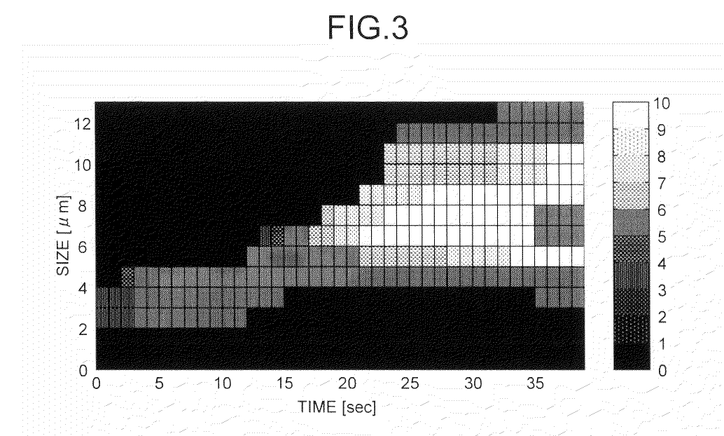

FIG. 3 is a drawing of Chart 1 according to the present embodiment. As the numerical value information generated by the thrombus detecting unit 25, the chart display unit 28a obtains quantities of thrombi corresponding to different sizes that are sectioned into predetermined time period units, from the storage unit 30. As mentioned above, the numerical value information may be processed, as necessary. Further, for example, the chart display unit 28a generates Chart 1 that indicates temporal changes (i.e., changes over the course of time) in a distribution of sizes of thrombi and the quantity of thrombi for each size, by preparing a chart in which time is assigned to the horizontal axis, the size of thrombi is assigned to the vertical axis, and colors are assigned to the quantity of thrombi and by plotting the numerical value information generated by the thrombus detecting unit 25 into the prepared chart. For example, the colors are displayed in colors of ten different levels and assigned in such a manner that, for example, the higher the brightness level is, the larger is the quantity of thrombi; and the lower the brightness level is, the smaller is the quantity of thrombi.

Chart 1 shown in FIG. 3 indicates in what size and in what quantity, approximately, the thrombi were present at a certain point in time and how the status changed over the course of time. Further, in Chart 1, if the width of a high-brightness region is large at a certain point in time, it means that that the range of the distribution of sizes of thrombi is large, i.e., thrombi of many different sizes were present at that point in time. Further, in Chart 1, the moving of the high-brightness region over the course of time indicates peak time periods for the presence of thrombi. For the sake of convenience in the explanation, the colors are expressed with gradations in FIG. 3; however, the colors may be expressed with different colors or expressed with gradations.

Next, the significance of displaying the chart that indicates a distribution of sizes of thrombi will be explained. The risk levels are not the same for a situation where a large quantity of small-sized thrombi are present and for a situation where a large quantity of large-sized thrombi are present. For example, if a large quantity of large-sized thrombi are present, it is conjectured that there is a high possibility that the large-sized thrombi will cause a blockage in a relatively large blood vessel or at a narrow part of a large vessel, before small-sized thrombi cause a blockage in peripheral blood vessels. As explained here, it is useful to display the chart that indicates a distribution of sizes of the thrombi.

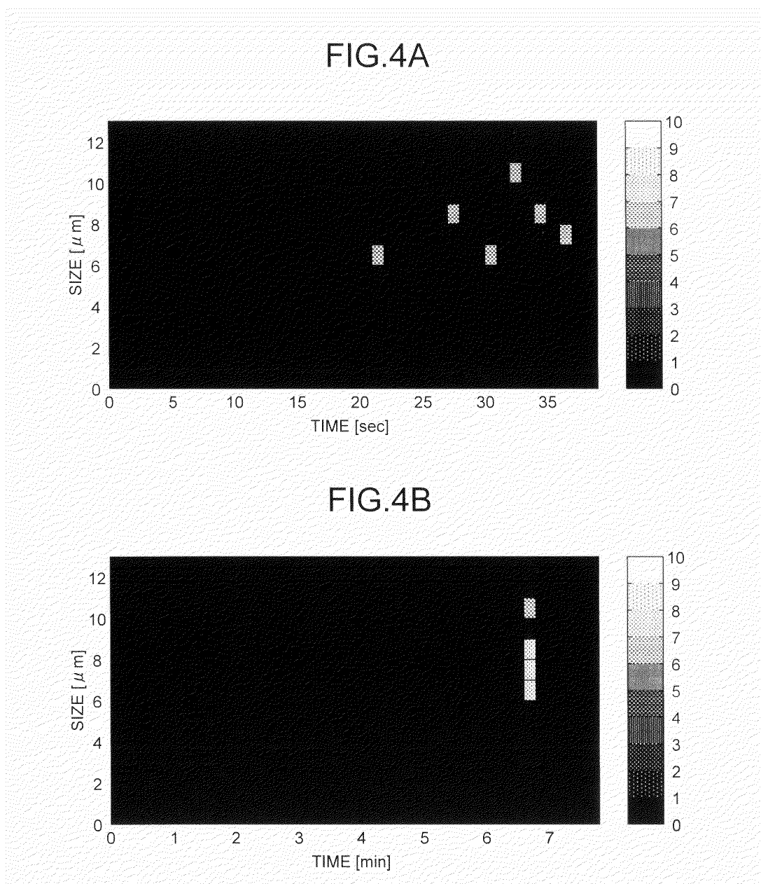

FIGS. 4A and 4B are drawings of modification examples of Chart 1 according to the present embodiment. As shown in FIGS. 4A and 4B, the chart display unit 28a according to the present embodiment changes, as appropriate, the scale along the time-axis direction, in accordance with thrombus detection statuses. For example, let us discuss a situation where, as shown in FIG. 4A, the chart display unit 28a is displaying temporal changes during a time period of 40 seconds, while using a scale with graduations in units of seconds. In this situation, for example, if only a small quantity of thrombi are detected during a time period of one minute, the high-brightness regions are dispersed in Chart 1, as shown in FIG. 4A, and it is difficult to understand the whole picture of the thrombi that are present in the blood. To cope with this situation, the chart display unit 28a changes the scale on the horizontal axis to a scale using longer time units (e.g., graduations in units of minutes), as shown in FIG. 4B. For example, it is a good idea to determine, in advance, a threshold value (e.g., a quantity of thrombi detected during a time period of one minute) that is used as a reference for changing the scale, so that the thrombus detecting unit 25 compares a thrombus detection status with the threshold value and instructs the chart display unit 28a to change the scale if, as a result of the comparison, only such a quantity of thrombi that is smaller than the threshold value is detected.

Possible embodiments of Chart 1 are not limited to the examples described above. For example, the change can be made with predetermined timing, instead of being automatically made in accordance with the thrombus detection statuses. Further, for example, it is also acceptable to display Chart 1 shown in FIG. 4A side by side with Chart 1 shown in FIG. 4B on the display unit 14, instead of changing the scale.

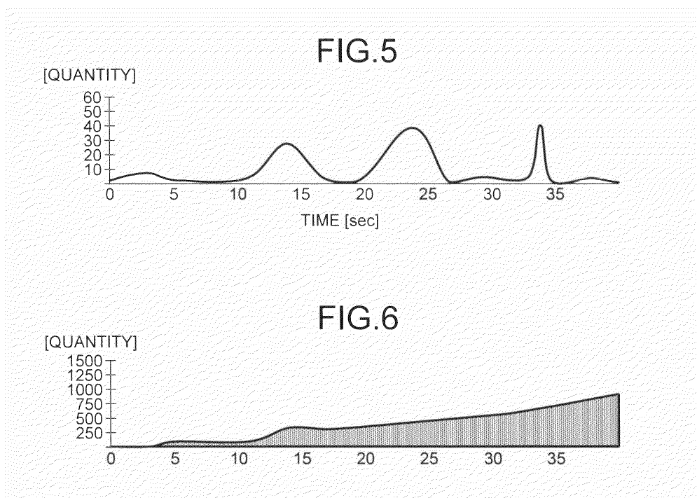

FIG. 5 is a drawing of Chart 2 according to the present embodiment. As the numerical value information generated by the thrombus detecting unit 25, the chart display unit 28a obtains quantities of thrombi that are sectioned into predetermined time period units, from the storage unit 30. After that, for example, the chart display unit 28a generates Chart 2 that indicates temporal changes in the quantity of thrombi, by preparing a chart in which time is assigned to the horizontal axis, whereas the quantity of thrombi is assigned to the vertical axis and by plotting the numerical value information generated by the thrombus detecting unit 25 into the prepared chart. Chart 2 shown in FIG. 5 indicates instantaneous values of the detected quantities of thrombi.

Possible embodiments of Chart 2 are not limited to the example described above. For instance, the chart display unit 28a may obtain amounts of thrombi that are sectioned into predetermined time period units, as the numerical value information generated by the thrombus detecting unit 25. In this situation, the amount of thrombi is information about the total area of all the thrombi. Further, for example, the chart display unit 28a generates Chart 2 that indicates temporal changes in the amount of thrombi, by preparing a chart in which time is assigned to the horizontal axis, whereas the amount of thrombi is assigned to the vertical axis and by plotting the numerical value information generated by the thrombus detecting unit 25 into the prepared chart. It is also acceptable to apply the change of the scale to Chart 2, in the same manner as to Chart 1.

FIG. 6 is a drawing of Chart 3 according to the present embodiment. As the numerical value information generated by the thrombus detecting unit 25, the chart display unit 28a obtains an accumulated value of quantities of thrombi that are sectioned into predetermined time period units, from the storage unit 30. After that, for example, the chart display unit 28a generates Chart 3 that indicates temporal changes in the accumulated value of quantities of thrombi, by preparing a chart in which time is assigned to the horizontal axis, whereas the quantity of thrombi is assigned to the vertical axis and by plotting the numerical value information generated by the thrombus detecting unit 25 into the prepared chart. Chart 3 shown in FIG. 6 indicates quantities of thrombi that started being accumulated at a point in time. Chart 3 is useful in a situation where, for example, a thrombus formation state needs to be monitored during a surgery procedure that lasts for a long period of time. It is also acceptable to apply the change of the scale to Chart 3, in the same manner as to Chart 1. In addition, it is also acceptable to configure Chart 3 so as to indicate an accumulated value of amounts of thrombi, like in Chart 2.

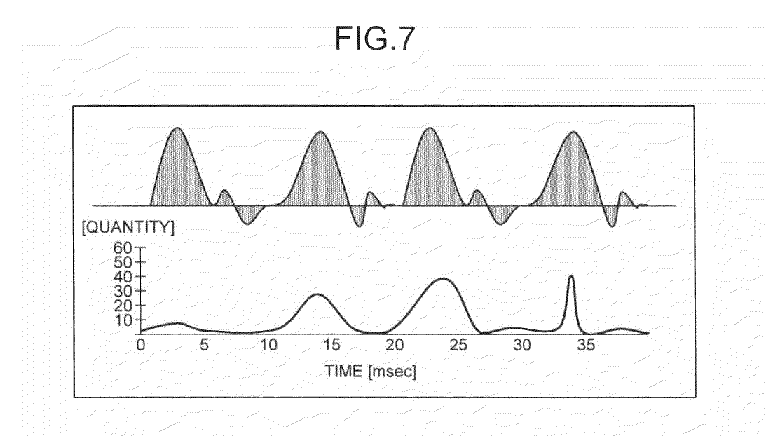

FIG. 7 is a drawing of Chart 4 according to the present embodiment. The chart display unit 28a obtains the numerical value information generated by the thrombus detecting unit 25 from the storage unit 30, and also receives a Doppler waveform generated by the image generating unit 26 in a real-time manner. The chart display unit 28a then displays the chart side by side with the Doppler waveform, in such a manner that the chart and the Doppler waveform are in synchronization with each other on the time axis. The Doppler waveform is a waveform indicating a flow rate of the blood and is generated by the image generating unit 26 on the basis of the Doppler data generated by the Doppler processing unit 24. For example, the image generating unit 26 outputs Doppler shift amounts with respect to a sample volume set in a B-mode image, in a time-series manner. For example, as shown in FIG. 7, the chart display unit 28a displays the chart indicating the temporal changes in the quantity of thrombi side by side with the Doppler waveform, in such a manner that the scales thereof on the horizontal axis match each other.

When the chart indicating the thrombus detection result is displayed side by side with the Doppler waveform in this manner, it is desirable to configure the thrombus detecting unit 25 so as to use a method by which thrombi are detected from Doppler data, as the method for detecting the thrombi. The reason can be explained as follows: For example, let us assume that a series of routines carried out in an actual medical situation includes a procedure of acquiring Doppler data and displaying a Doppler waveform. In that situation, if the thrombus detecting unit 25 is configured to use a method by which thrombi are detected from Doppler data as the method for detecting the thrombi, it is possible to generate both a chart indicating a thrombus detection result and a Doppler waveform from the single piece of Doppler data. Also, in this situation, the designation of a region received by the receiving unit 28b in the B-mode image may be used by both the thrombus detecting unit 25 and the Doppler processing unit 24. In other words, the thrombus detecting unit 25 can detect thrombi in the designated region, whereas the Doppler processing unit 24 can output the Doppler waveform indicating the flow rate of the bloodstream in the region while using the region as a sample volume.

When making a diagnosis about the presence of thrombi, it is desirable to make an evaluation in association with failures in the motions of the heart and failures in the functions of the auxiliary artificial heart. Thus, because Chart 4 indicates the temporal changes of the thrombi together with the Doppler waveform, Chart 4 is useful. Further, Chart 4 is also useful in a situation where, for example, the user wishes to observe a flow at the moment when thrombi float around. For example, let us assume that each of the parts of a Doppler waveform having a large amplitude indicates a temporal phase with fast bloodstream (a systolic period), whereas each of the parts of a Doppler waveform having a small amplitude indicates a temporal phase with slow bloodstream (a diastolic period). In that situation, if the thrombi are light, instantaneous values of the quantity of thrombi exhibit a tendency similar to that of the Doppler waveform in synchronization therewith. For example, it is considered that, in each of the parts of the Doppler waveform having a large amplitude, the instantaneous value of the quantity of thrombi also becomes large, whereas in each of the parts of the Doppler waveform having a small amplitude, the instantaneous value of the quantity of thrombi also becomes small. In contrast, if the thrombi are heavy, it is considered that the instantaneous value of the quantity of thrombi is not in synchronization with the Doppler waveform.

Possible embodiments of Chart 4 are not limited to the example described above. For example, it is acceptable even if the time range of the Doppler waveform is not equal to the time range of the chart. For instance, it is acceptable to arrange the time range of the chart to be larger than the time range of the Doppler waveform. For example, it is acceptable to configure the chart so as to display all the temporal changes from the beginning of the measuring process.

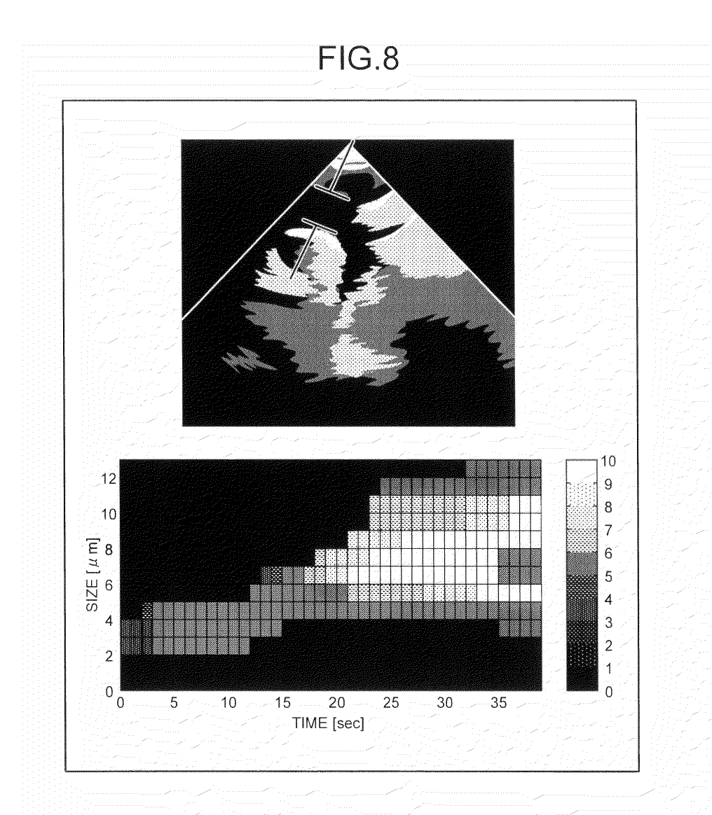

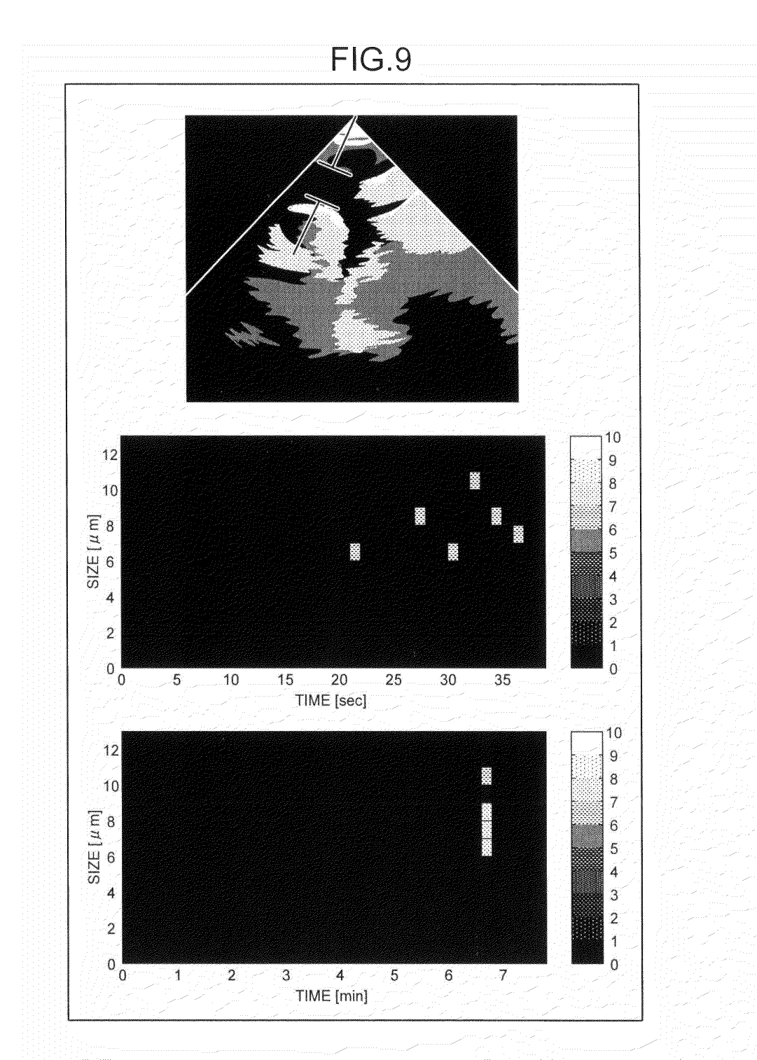

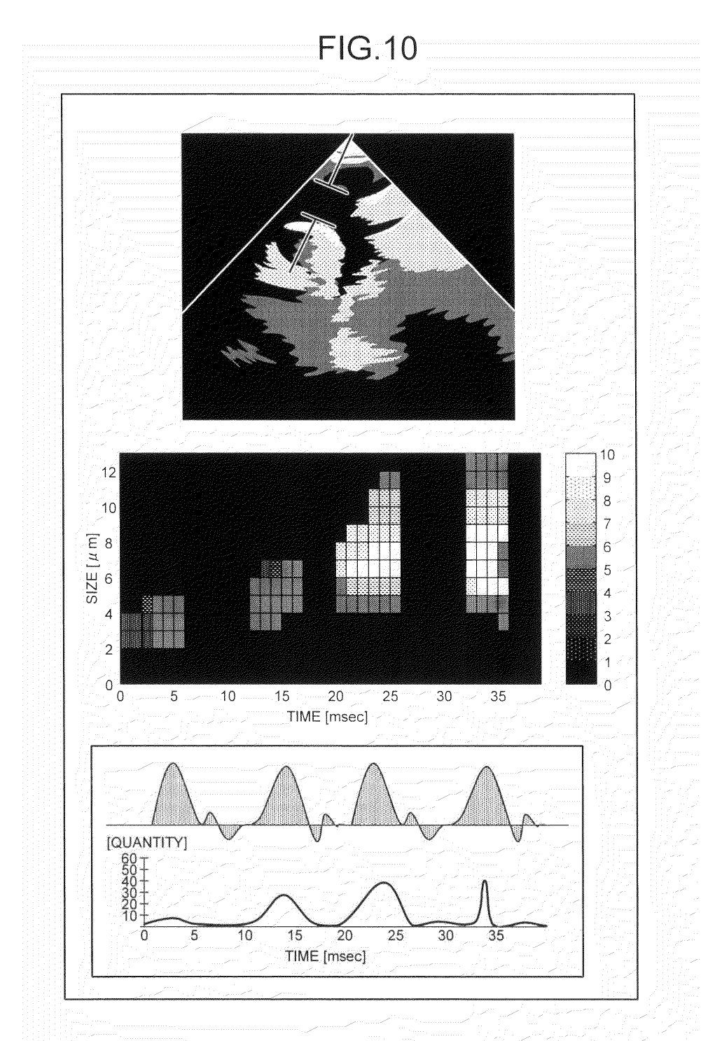

FIGS. 8 to 10 are drawings of Chart 5 according to the present embodiment. For example, as shown in FIG. 8, the chart display unit 28a may display the B-mode image used for setting the analysis region side by side with Chart 1. In another example, as shown in FIG. 9, the chart display unit 28a may display the B-mode image used for setting the analysis region side by side with two types of Chart 1 using mutually-different scales. In yet another example, as shown in FIG. 10, the chart display unit 28a may display the B-mode image used for setting the analysis region side by side with Chart 1, a Doppler waveform, and Chart 2. In examples other than those illustrated in FIGS. 8 to 10, the chart display unit 28a may also display any of the various types of charts shown in FIGS. 2 to 7 in combination as appropriate. Further, the chart display unit 28a may display a B-mode image, an M-mode image, or a Doppler image acquired in a real-time manner, instead of the B-mode image used for setting the analysis region.

As explained above, according to at least one aspect of the present embodiment, it is possible to properly monitor the thrombi. First, the method makes it possible to conveniently monitor the thrombi from the subject's body surface, without inserting the ultrasound probe 12 into the subject's body. In addition, because the ultrasound diagnosis apparatus 100 is configured to quantitatively (or semi-quantitatively) present the thrombus formation state of the subject P to the user, the user is able to properly monitor the thrombi. For example, the ultrasound diagnosis apparatus 100 detects the thrombi that are present in the blood of the subject from the echo signals and displays the chart (e.g., Chart 1 shown in FIG. 3) indicating the temporal changes in the distribution of the sizes of the thrombi and the quantity of thrombi for each of the sizes. The chart provides the user with the information that serves as an index used for selecting an appropriate treatment method.

Furthermore, according to at least one aspect of the present embodiment, the chart indicating the thrombus detection result is displayed side by side with the Doppler waveform in such a manner that the chart is in synchronization with the Doppler waveform on the time axis. Thus, for example, the user is able to understand the thrombus formation state in association with the state of the bloodstream.

Although some of the exemplary embodiments have thus been explained, possible embodiments are not limited to those.

In the embodiments described above, the thrombus detecting unit 25 is described as being configured to generate the numerical value information that quantifies the thrombus detection result; however, possible embodiments are not limited to this example. It is acceptable to configure the thrombus detecting unit 25 so as to further generate an image that clearly indicates the detected thrombi (e.g., an image obtained by assigning a color to the pixels corresponding to the thrombi and superimposing the color-assigned image onto a B-mode image) and to send the generated image to the storage unit 30. In that situation, for example, the chart display unit 28a may obtain the image generated by the thrombus detecting unit 25 by referring to the storage unit 30 and may display the obtained image side by side with the display of a chart indicating the thrombus detection result. Further, in another example, the display processing unit 28 may display the image generated by the thrombus detecting unit 25 side by side with the display of a B-mode image or a Doppler image.

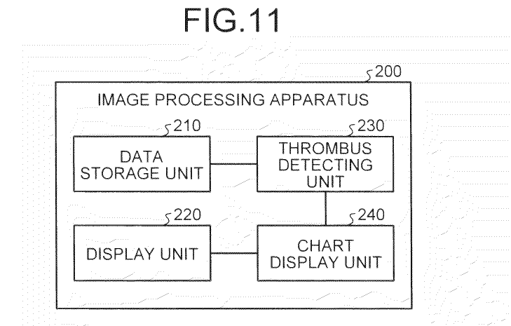

Further, FIG. 11 is a diagram of an image processing apparatus 200 according to another embodiment. In the embodiments described above, the ultrasound diagnosis apparatus 100 is described as including the thrombus detecting unit 25 and the chart display unit 28a. However, possible embodiments are not limited to this example. The processes performed by the thrombus detecting unit 25 and the processes performed by the chart display unit 28a do not necessarily have to be executed in the ultrasound diagnosis apparatus 100 and may be executed in the image processing apparatus 200, which is provided as a separate component. As shown in FIG. 11, the image processing apparatus 200 includes a data storage unit 210, a display unit 220, a thrombus detecting unit 230, and a chart display unit 240.

For example, by receiving data from the ultrasound diagnosis apparatus 100 via a network or by receiving an input from the operator, the data storage unit 210 stores therein B-mode data or Doppler data on which the analyzing process is to be performed. The thrombus detecting unit 230 is configured to detect thrombi, if any, that are present in the blood of the subject, from the B-mode data or the Doppler data stored in the data storage unit 210 and to generate numerical value information that quantifies the thrombus detection result. Further, the chart display unit 240 is configured to display a chart indicating the thrombus detection result on the display unit 220, on the basis of the numerical value information. In addition, the thrombus detecting unit 230 is configured to perform the same processes as those performed by the thrombus detecting unit 25 described in the exemplary embodiments above. The chart display unit 240 is also configured to perform the same processes as those performed by the chart display unit 28a described in the exemplary embodiments above.

In the embodiments described above, the example is explained in which the thrombi are detected at the aortic arch, while using the approach by which the ultrasound probe is placed on the suprasternal notch without being inserted in the subject's body. However, possible embodiments are not limited to this example. For instance, another method may be used by which a transesophageal probe is inserted into the subject's body so as to detect thrombi flowing in an aorta or a pulmonary artery.

Further, the processing procedure performed by the thrombus detecting unit 25 and the processing procedure performed by the chart display unit 28a that are described in the exemplary embodiments above may be realized by causing a computer such as the controlling processor 29 to execute the “image processing program” stored in advance in the storage unit 30. The “image processing program” may be distributed via a network such as the Internet. Further, it is also possible to record the “image processing program” onto a computer-readable recording medium such as a hard disk, a flexible disk (FD), a Compact Disk Read-Only Memory (CD-ROM), a Magneto-optical (MO) disk, or a Digital Versatile Disk (DVD), so that a computer such as the controlling processor 29 reads the image processing program from the recording medium and executes the read image processing program.

By using the ultrasound diagnosis apparatus, the image processing apparatus, and the image processing computer program according to at least one aspect of the exemplary embodiments, it is possible to monitor the thrombi conveniently and properly.

While certain embodiments have been described, these embodiments have been presented by way of example only, and are not intended to limit the scope of the inventions. Indeed, the novel embodiment described herein may be embodied in a variety of other forms; furthermore, various omissions, substitutions and changes in the form of the embodiment described herein may be made without departing from the spirit of the inventions. The accompanying claims and their equivalents are intended to cover such forms or modifications as would fall within the scope and spirit of the inventions.

What is claimed is:

1. An ultrasound diagnosis apparatus comprising:
a transmitting and receiving unit configured to transmit an ultrasound pulse to a subject and to receive an echo signal from the subject;
a detecting unit configured to detect thrombi that are present in blood of the subject from the echo signal and to generate numerical value information that quantifies a thrombus detection result; and
a chart display unit configured to display a chart indicating the thrombus detection result on a display unit, on a basis of the numerical value information.

2. The ultrasound diagnosis apparatus according to claim 1, wherein the chart display unit displays the chart indicating changes in the thrombi over a course of time, on the basis of the numerical value information.

3. The ultrasound diagnosis apparatus according to claim 1, wherein the chart display unit changes a scale along a time-axis direction of the chart, in accordance with a status of the thrombus detection by the detecting unit.

4. The ultrasound diagnosis apparatus according to claim 2, wherein
the detecting unit detects a quantity of thrombi that are present in the blood of the subject from the echo signal and generates information about the quantity of thrombi as the numerical value information, and
the chart display unit displays at least one selected from the following: a chart indicating temporal changes in the quantity of thrombi; and a chart indicating temporal changes in an accumulated value of quantities of thrombi.

5. The ultrasound diagnosis apparatus according to claim 1, wherein
the detecting unit detects an area of each of the thrombi that are present in the blood of the subject from the echo signal and generates information about the area of each of the thrombi as the numerical value information, and
on a basis of the information about the area of each of the thrombi, the chart display unit displays a chart indicating a distribution of sizes of the thrombi.

6. The ultrasound diagnosis apparatus according to claim 1, further comprising: a receiving unit configured to receive a designation of a region in a B-mode image displayed on the display unit, wherein
the detecting unit detects the thrombi within the region.

7. The ultrasound diagnosis apparatus according to claim 2, further comprising: a Doppler processing unit configured to output a Doppler waveform that indicates a flow rate of the blood of the subject, wherein
the chart display unit displays the chart indicating the changes in the thrombi over the course of time side by side with the Doppler waveform, while the chart and the Doppler waveform are in synchronization with each other on a time axis.

8. The ultrasound diagnosis apparatus according to claim 7, further comprising: a receiving unit configured to receive a designation of a region in a B-mode image displayed on the display unit, wherein
the detecting unit detects the thrombi within the region, and
the Doppler processing unit outputs a Doppler waveform that indicates a flow rate of bloodstream within the region.

9. An image processing apparatus comprising:
a detecting unit configured to detect thrombi that are present in blood of a subject from an echo signal obtained by an ultrasound diagnosis apparatus and to generate numerical value information that quantifies a thrombus detection result; and
a chart display unit configured to display a chart indicating the thrombus detection result on a display unit, on a basis of the numerical value information.

10. The image processing apparatus according to claim 9, wherein the chart display unit displays the chart indicating changes in the thrombi over a course of time, on the basis of the numerical value information.

11. The image processing apparatus according to claim 9, wherein the chart display unit changes a scale along a time-axis direction of the chart, in accordance with a status of the thrombus detection by the detecting unit.

12. The image processing apparatus according to claim 10, wherein
the detecting unit detects a quantity of thrombi that are present in the blood of the subject from the echo signal and generates information about the quantity of thrombi as the numerical value information, and
the chart display unit displays at least one selected from the following: a chart indicating temporal changes in the quantity of thrombi; and a chart indicating temporal changes in an accumulated value of quantities of thrombi.

13. The image processing apparatus according to claim 9, wherein
the detecting unit detects an area of each of the thrombi that are present in the blood of the subject from the echo signal and generates information about the area of each of the thrombi as the numerical value information, and
on a basis of the information about the area of each of the thrombi, the chart display unit displays a chart indicating a distribution of sizes of the thrombi.

14. The image processing apparatus according to claim 9, further comprising: a receiving unit configured to receive a designation of a region in a B-mode image displayed on the display unit, wherein
the detecting unit detects the thrombi within the region.

15. The image processing apparatus according to claim 10, further comprising: a Doppler processing unit configured to output a Doppler waveform that indicates a flow rate of the blood of the subject, wherein
the chart display unit displays the chart indicating the changes in the thrombi over the course of time side by side with the Doppler waveform, while the chart and the Doppler waveform are in synchronization with each other on a time axis.

16. The image processing apparatus according to claim 15, further comprising: a receiving unit configured to receive a designation of a region in a B-mode image displayed on the display unit, wherein
the detecting unit detects the thrombi within the region, and
the Doppler processing unit outputs a Doppler waveform that indicates a flow rate of bloodstream within the region.

17. An image processing method comprising:
detecting thrombi that are present in blood of a subject from an echo signal obtained by an ultrasound diagnosis apparatus;
generating numerical value information that quantifies a thrombus detection result; and
displaying a chart indicating the thrombus detection result on a display unit, on a basis of the numerical value information.
